(19) United States
(12) Patent Application Publication
(10) Pub. No.: US 2001/0007660 A1
(43) Pub. Date: Jul. 12, 2001

(54) METHODS OF TREATING AND PROTECTING AGAINST TUBERCULOSIS USING A MONOCLONAL ANTIBODY SELECTIVE FOR MYCOBACTERIUM TUBERCULOSIS

(76) Inventors: AHARONA GLATMAN-FREEDMAN, IRVINGTON, NY (US); ARTURO CASADEVALL, PELHAM, NY (US)

Correspondence Address: AMSTER ROTHSTEIN AND EBENSTEIN, 90 PARK AVENUE, NEW YORK, NY, 10016

(21) Appl. No.: 08/868,545

(22) Filed: Jun. 4, 1997

(51) Int. Cl. A61K 39/40; A61K 39/395

(52) U.S. Cl. 424/168.1; 424/130.1

(57) ABSTRACT

The present invention is directed to compositions comprising a monoclonal antibody that reacts with surface epitopes of M. tuberculosis, methods of treating tuberculosis by passively immunizing a subject using the antibody compositions, antigenic determinants for use as a vaccine to protect against M. tuberculosis infection, and a method of using the vaccine to prevent infections of M. tuberculosis.

### STATEMENT OF GOVERNMENT INTEREST

This invention was made with government support under NIH Training Grant No. 1 T32 AI07501-01, and NIH Grant Nos. AI-33774 and AI-33142. As such, the government has certain rights in this invention.

### BACKGROUND OF THE INVENTION

Tuberculosis continues to be a major worldwide health problem and is responsible for most incidences of death by an infectious agent. The worldwide incidence of tuberculosis was estimated by the World Health Organization to be 8.8 million in 1995, with a mortality estimate of 3.0 million persons, and is expected to rise to 10.2 million by the year 2000 (Dolin, et al., Bull. WHO. 72:213-220 (1994)). The tuberculosis problem has been compounded by the development of the AIDS epidemic and the growing number of HIV-related cases of tuberculosis (Dolin, et al., Bull. WHO. 72:213-220 (1994)). Effective treatment of tuberculosis is generally prolonged, especially in patients also infected with HIV. In the past, infection with drug-sensitive strains of the M. tuberculosis complex had been cured with certain antibiotics, including isoniazid, rifampicin, ethionamide and pyrazinamide. However, resistance to isoniazid and other antibiotics has developed in many strains of M. tuberculosis. The only licensed vaccine, the BCG vaccine, is controversial in regard to its efficacy, and its effectiveness varies markedly from country to country. This has resulted in the continued search for an effective vaccine against M. tuberculosis.

Mycobacterium tuberculosis is an intracellular pathogen, thought not to be reached by antibody immunity. Recent studies suggest that some IgA antibodies can neutralize viruses inside cells (Mazanec, et al. 1992) and that monoclonal antibodies inhibit intracellular Toxoplasma gondii (Mineo, et al. 1994). It is known that patients with tuberculosis mount high levels of serum antibodies (Favez, et al. 1966). This antibody response is polyclonal and may contain protective, non-protective, and enhancing antibodies. In such a case, monoclonal antibody technology can be used to identify the protective antibodies. Passive antibody therapy was used to treat tuberculosis in the pre-antibiotic era in the form of serum therapy. The results of that treatment were equivocal, but some investigators published positive results (Maragliano 1896, Paquin 1895, and Marmorek 1903). Since that time, antimicrobial therapy has been the only treatment available for tuberculosis. The rise in antimicrobial resistance, however, has created a sense of urgency for the development of alternative methods of therapy for tuberculosis.

### SUMMARY OF THE INVENTION

The present invention provides for compositions comprising a monoclonal antibody that reacts with a surface epitope of M. tuberculosis, methods of treating tuberculosis by passively immunizing a subject using the antibody composition, and antigenic determinates for use as a vaccine to protect against M. tuberculosis infection.

### BRIEF DESCRIPTION OF THE FIGURES

FIG. 1: FIG. 1 sets forth the binding of monoclonal antibodies 5c11, 4f11, and 9d8 to M. tuberculosis whole cell ELISA at various concentrations. The diagram shows ELISA configuration.

FIGS. 2A and 2B: FIG. 2 sets forth the double staining of M. tuberculosis by acid-fast staining and immunofluorescence (shown here as a clump) with monoclonal antibody 5c11 at a concentration of 10 μg/ml. FIG. 2A: Acid-fast staining. FIG. 2B: Indirect immunofluorescence. Immunostaining with monoclonal antibodies 4f11 and 9d8 produced similar fluorescence (not shown). Bar=10 μm. The picture was generated using Kodak RFS 2035 scanner and Adobe Photoshop version 3.0 for Macintosh.

FIGS. 3A, 3B and 3C: FIGS. 3A-3C represent immunoelectronmicroscopy demonstrating the binding of monoclonal antibodies to M. tuberculosis. Gold particles denote secondary antibody binding to the primary monoclonal antibody. FIGS. 3A, 3B, and 3C correspond to monoclonal antibodies 5c11, 4f11 and 9d8, respectively. Bar=0.2 μm.

FIGS. 4A, 4B, and 4C: FIGS. 4A-4C show the binding of monoclonal antibodies 5c11, 4f11 and 9d8 with and without sodium meta-periodate treatment by whole cell ELISA. Filled symbols correspond to monoclonal antibody binding to periodate treated mycobacteria, whereas open symbols correspond to non-periodate treated mycobacteria. FIGS. 4A, 4B, and 4C correspond to monoclonal antibodies 5c11, 4f11 and 9d8, respectively. Diagram shows the ELISA configuration.

FIGS. 5A, 5B, and 5C: FIGS. 5A-5C show the binding of monoclonal antibodies at various concentrations to mycobacterial surface carbohydrates. FIG. 5A: Binding of 5c11, 4f11 and 9d8 to LAM. FIG. 5B: Binding of 5c11, 4f11 and 9d8 to mAGP. FIG. 5C: Comparative binding of 5c11 to LAM and LM at antigen concentration of 1 μg/ml. The diagram shows the ELISA configuration.

FIGS. 6A, 6B, and 6C: FIGS. 6A-6C set forth the binding of monoclonal antibodies to M. tuberculosis using whole cell ELISA with and without pre-treatment with proteinase K treatment. Filled symbols correspond to monoclonal antibody binding to proteinase K treated mycobacteria, whereas open symbols correspond to non-proteinase K treated mycobacteria. FIGS. 6A, 6B, and 6C correspond to monoclonal antibodies 5c11, 4f11 and 9d8, respectively. The diagram shows the ELISA configuration.

FIGS. 7A and 7B: FIG. 7A demonstrates lung tissue from a mouse that received NSO ascites (Group 1). Acid-fast bacilli are dispersed throughout the tissue. Cells are not well organized in granulomas. FIG. 7B shows lung tissue from a mouse that received a mixture of M. tuberculosis and monoclonal antibody 9d8 (Group 2). Acid fast bacilli are contained in well-organized granulomas. The best organization is represented by few layers of cells circumscribing acid-fast bacilli.

FIG. 8: FIG. 8 sets forth survival data of the efficacy of a monoclonal antibody against M. tuberculosis in prolonging survival in a murine respiratory challenge model. Mice treated with a mixture of M. tuberculosis and monoclonal antibody (round symbols) survived longer than mice from the other two groups. Mice treated with NSO ascites and 9d8 ascites intraperitoneally died by day 2.

### DETAILED DESCRIPTION OF THE INVENTION

The present invention is directed to compositions comprising a monoclonal antibody that reacts with a surface epitope of M. tuberculosis, methods of treating tuberculosis by passively immunizing a subject using the antibody compositions, antigenic determinates for use as a vaccine to protect against M. tuberculosis infection, and a method of using the vaccine to prevent infections of M. tuberculosis.

The hybridoma cell lines provided by the present invention were obtained by immunizing mice with whole cells of M. tuberculosis using methods commonly know to those skilled in the art. Spleen cells were obtained from mice exhibiting a positive antibody reaction and fused with myeloma cells to obtain hybridoma cells which secrete monoclonal antibodies against the cell wall of M. tuberculosis. The hybridoma cell lines which secrete these antibodies are herein designated H-4f11, H-5c11, and H-9d8. H-9d8 has been deposited in the American Type Culture Collection (ATCC), 12301 Parklawn Drive, Rockville, Md., 20852, on Jun. 3, 1997, under ATCC Accession Number ______. This deposit was made pursuant to and in satisfaction of the Budapest Treaty on the International Recognition of the Deposit of Microorganisms for the Purposes of Patent Procedure. The hybridoma cell lines provided by the present invention may be fused with other cells to transfer the genes which express the monoclonal antibodies, thus providing new hybridomas.

The monoclonal antibodies generated by these hybridoma cell lines are herein designated 4f11, 5c11, and 9d8. These antibodies were characterized by ELISA, indirect immunofluorescence and immunoelectronmicroscopy. The antibodies, however, may be purified by any convenient techniques, such as chromatography, electrophoresis, precipitation, and extraction. Monoclonal antibody 4f11 recognizes a cell wall carbohydrate that belongs to the mycolyl-arabinogalactanpeptidoglycan (mAGP) complex of mycobacteria, monoclonal antibody 5c11 binds lipoarabinomannan (LAM), and monoclonal antibody 9d8 selectively binds to a non-protein cell surface epitope of M. tuberculosis. The above-described antibodies and hybridoma cell lines are discussed in Application No. ______, filed Jun. 4, 1997, entitled “Monoclonal Antibodies to Mycobacterium Tuberculosis and a Modified ELISA Assay”, which is herein incorporated by reference.

The present invention also provides for humanized antibodies of monoclonal antibody 9d8. Humanized antibodies are synthetic molecules composed of human antibody protein sequences that retain the antigen-binding site of the heterologous antibody, and are produced by methods commonly know to those skilled in the art. Specifically, murine monoclonal antibody 9d8 may be converted to a humanized antibody by obtaining the mouse variable regions specific for the antigen from the H-9d8 hybridoma and then joined by recombinant DNA techniques to human constant regions, which are usually obtained from genetic clones. The resulting chimeric genes are then transfected into a recipient cell line and the transfected cell lines synthesizing functional antibodies are identified and isolated for in vivo or in vitro amplification.

The monoclonal antibody provided by the present invention may be used to passively immunize individuals against tuberculosis. The protective antibodies produced may be isolated and used as a therapeutic antibody in a manner analogous to the current use of immune or hyperimmune globulin preparations. The immune or hyperimmune globulin may then be passively administered to a subject in need of such protective antibodies.

For passive immunization, the monoclonal antibody of the present invention is administered in conjunction with a suitable pharmaceutical carrier. Representative examples of suitable carriers include, but are not limited to, distilled water, physiological saline, and phosphate-buffered aqueous solution. Vehicles are well known in the art and the selection of a suitable vehicle is deemed to be within the scope of those skilled in the art from the teachings contained herein. The selection of a suitable vehicle is also dependent on the manner in which the antibodies are to be administered. Adjuvants, such as Freund's adjuvant, complete or incomplete, may be added to enhance antigenicity. Other non-limiting examples of adjuvants include aluminum hydroxide, aluminum phosphate, calcium phosphate, beryllium hydroxide, and alum.

To inoculate a subject, the monoclonal antibody may be in the form of an injectable dose, and may be administered intramuscularly, subcutaneously, orally, intranasally, intravenously, or as an aerosol. The amount required may vary, and need only be an amount sufficient to induce a passive immune response typical of that obtained by the administration of antibodies. There may be a number of injections, using techniques known to one skilled in the art. Preferably the monoclonal antibody is administered early in the course of the disease.

The present invention further provides for antigenic determinants recognized by the monoclonal antibody for use as a vaccine. These antigenic determinants have the same immunogenic activity of the cell surface epitope antigen of M. tuberculosis to which monoclonal antibody 9d8 binds. This surface epitope protein of M. tuberculosis may be identified and characterized using procedures known to one skilled in the art. From this point, the antigenic determinant may be synthesized for use as a vaccine.

The degree of binding of the antigenic determinants of the present invention to human antibodies is determined by methods known to one skilled in the art. Specifically, human subjects are vaccinated with a vaccine to M. tuberculosis, and a subject is chosen who has a high titer of antibodies against M. tuberculosis. Specimens of pre-vaccination and post-vaccination serum is removed from said subject, and the M. tuberculosis antibodies are isolated from the serum. The antibodies may be isolated by methods commonly known to one skilled in the art, and may include procedures such as immunoprecipitation, gel filtration, ion exchange chromatography, and affinity chromatography. The affinity chromatography procedure may use M. tuberculosis coupled to sepharose. Pure antibody is eluted from the immunoabsorbant with chaotropic agents such as sodium thiocyanate, glycine-HCL buffer, or diethylamine buffer. After isolation of the antibodies, it is then determined whether the human antibodies bind to the antigenic determinants of the present invention, using common methods known to those skilled in the art.

The antigenic determinants of the present invention may be coupled with a conjugate for a more effective vaccine. Non-limiting examples of conjugates to which the antigenic determinants may be coupled include exotoxins, such as tetanus toxoid, diphtheria, and exotoxin A, and inactivated toxins, such as formalin.

To form a vaccine, the antigenic determinants of the present invention are administered in conjunction with a suitable pharmaceutical carrier. Representative examples of suitable carriers include, but are not limited to, distilled water, physiological saline, and phosphate-buffered aqueous solution. Vehicles for vaccines are well known in the art and the selection of a suitable vehicle is deemed to be within the scope of those skilled in the art from the teachings contained herein. The selection of a suitable vehicle is also dependent on the manner in which the vaccine is to be administered. Non-limiting examples of adjuvants include aluminum hydroxide, aluminum phosphate, calcium phosphate, beryllium hydroxide, and alum. The amount of adjuvant may be chosen from the range of amounts that are necessary for increasing antigenicity.

To inoculate a subject, the vaccine may be in the form of an injectable dose, and may be administered intramuscularly, subcutaneously, orally, intranasally or intravenously. In one embodiment of the invention, the vaccine is administered as an aerosol. The amount required may vary, and need only be an amount sufficient to induce an immune response typical of existing vaccines. There would typically be two injections, one primary and one booster, using vaccination techniques known to one skilled in the art. The booster immunizations may be repeated as needed.

The present invention is described in the following Experimental Details Section, which is set forth to aid in an understanding of the invention, and should not be construed to limit in any way the invention as defined in the claims which follow thereafter.

Experimental Details Section

A. Materials and Methods

M. tuberculosis for immunization and hybridoma testing. M. tuberculosis Erdman strain was obtained from Trudeau Mycobacterial Culture Collection, Trudeau Institute, Saranac Lake, N.Y. (TMC 107) and grown in Proskauer-Beck- Trudeau (PBT) medium without Tween at 37° C. for 5 weeks. Mycobacterial cells were washed twice in phosphate-buffered-saline (PBS), heat inactivated at 80° C. for 2 h and sonicated for 3 to 5 seconds (Branson Ultrasonics, Danbury, Conn.).

Mycobacterial strains for cross reactivity testing. Mycobacterial strains used in this study originated from the American Type Culture Collection, Rockville, Md. (ATCC), Trudeau Mycobacterial Culture Collection, Trudeau Institute, Saranac Lake, N.Y. (TMC), Centers for Disease Control, Atlanta, Ga. (CDC), NY Department of Health (NY DOH), P. D'Arcy Hart (PDH) and College of American Pathologists, Northfield, Ill. (CAP). M. tuberculosis (TMC 107), M. microti (PDH), M. bovis-BCG (Pasteur Institute), M. avium (CAP—Inderlied 101), M. smegmatis (CDC), M. xenopi (ATCC 19250), M. chitae (ATCC 19627), M. marinum (ATCC 927), M. chelonae (CDC), M. gastri (ATCC 25028), M. kansasii (ATCC 12478), M. vaccae (CDC), M. phlei (TMC 1516), M. fortuitum (ATCC 6841), M. terrae (ATCC 15755), M. szulgai (ATCC 35799) and M. gordonae (ATCC 14470) were grown in Lowenstein-Jensen (LJ) slants. Several bacterial species were obtained as well: Streptococcus pneumoniae, Escherichia coli, Corynebacterium pseudodiphtheria, Pseudomonas aeruginosa, Haemophilus influenzae (quality control strains obtained from the Clinical Microbiology Laboratory, Montefiore Medical Center, Bronx, N.Y.) and Nocardia asteroides (clinical isolate, Mycology Laboratory, Montefiore Medical Center, Bronx, N.Y.). Cells were obtained from the media surface using a sterile loop, suspended in PBS with 0.1 mM sodium azide, sonicated briefly as described above to break clumps (when needed) and heat treated at 80° C. for 2 h.

M. tuberculosis whole cell ELISA. A 50 μl suspension of 1-2×10⁷ M. tuberculosis suspended in phosphate-buffered-saline (PBS) pH 7.2 was placed in microtiter ELISA plate wells and incubated at room temperature for 2 h. Prior to use in ELISA the M. tuberculosis suspension was briefly sonicated as described above. Plates were blocked with 1% bovine serum albumin (BSA) and 0.05% horse serum in PBS and stored at 4° C. Plates were washed 3 times with 0.05% Tween 20 in PBS. Hybridoma cell supernatants containing monoclonal antibodies were added to each well and the plates were incubated 1-1.5 h at 37° C. or overnight at 4° C. Plates were then washed 3 times, and 1 μg/ml goat anti-mouse alkaline phosphatase conjugated antibody (Southern Biotechnology Associates, Inc. Birmingham, Ala.) was added to each well and incubated 1-1.5 h at 37° C. After washing 5 times, a solution of 1 mg/ml p-nitrophenyl phosphate (Southern Biotechnology Associates, Inc. Birmingham, Ala.) in substrate buffer (0.001 M MgCl₂, 0.05 M Na₂CO₃, pH 9.8) was added (50 μl/well) and absorbance was measured at 405 nm in a Ceres 900 HDi reader (Bio-Tek Instruments Inc. Winooski, Vt.). ELISA measurements were the average of 3 microtiter wells.

Immunization. Balb/c mice (Jackson Laboratories, Bar Harbor, Me.) were injected intraperitoneally (i.p.) with approximately 2×10⁹ M. tuberculosis Erdman strain in an emulsion with incomplete Freund adjuvant (0.2 ml per mouse). The mice were boosted every 12-18 d for a period of seven weeks with 4.4×10⁷ to 1×10⁹ organism. Several booster injections included incomplete Freund adjuvant. Serum was examined for antibodies to M. tuberculosis by whole cell ELISA, and the mouse with the highest titer rise was boosted 4 d prior to fusion using 1×10⁹ organisms in incomplete Freund's Adjuvant.

Fusion. Spleen cells were harvested on day 50, fused with NSO myeloma cells at a ratio of 4:1 and suspended in HAT media. A total of 12 plates were seeded with fusion products and incubated at 37° C. with 10% CO₂. Hybridoma supernatants were screened for antibody production by whole cell ELISA.

Indirect Immunofluorescence (IF). This method was adapted from Jones et al. 1964 (Jones, et al. Am. Rev. Respir. Dis. 92:255-260 (1965)). Approximately 1×10⁷ heat killed M. tuberculosis were placed on a poly-L-lysine coated glass microscope slide (Poly-Prep slides, Sigma Diagnostics, St. Louis, Mo.) and fixed by heating at 65° C. for 2 h. Primary antibody was added at concentrations of 10, 1, 0.1, 0.01, 0.01 μg/ml and the slides were incubated for 30 min at room temperature. The slides were then washed with distilled water and incubated with FITC labeled anti mouse IgM or IgG (Southern Biotechnology Associates, Inc. Birmingham, Ala.) at a concentration of 10 μg/ml for 30 minutes at room temperature and without light. The slides were washed again with distilled water and sealed with mounting media (1.4 g glycine, 0.07 g NaOH, 1.7 g NaCl, 0.1 g sodium azide in 100 ml of distilled water, pH 8.6) with 1% n-propyl gallate. As a positive control, separate slides with M. tuberculosis cells were stained with acid-fast staining prior to indirect immunofluorescence. Negative controls consisted of Cryptococcus neoformans cells incubated with anti-M. tuberculosis antibodies, and M. tuberculosis incubated with anticryptococcal monoclonal antibodies of the same isotype. An additional negative control consisted of incubation of M. tuberculosis with FITC labeled antibodies.

Immunoelectronmicroscopy. A small pellet of heat killed M. tuberculosis was incubated in a microcentrifuge tube with 10 μg/ml monoclonal antibody in 1% BSA in PBS for 1 h at room temperature in a slow shaking motion. Cells were washed twice with PBS and incubated with gold labeled goat anti-mouse IgM+IgG (Amersham Life Science, Buckinghamshire, England), and diluted 1:30 in 1% BSA in PBS using the same conditions as above. Cells were then washed and fixed in Trump's fixative solution (4% paraformaldehyde and 1% glutaraldehyde in 0.1 M phosphate buffer at pH 7.3) overnight. Post fixation was done with 2% osmium for 1 h. Afterwards the cells were washed in 0.1 M phosphate buffer (pH 7.3) and dehydrated by incubation in solutions with increasing ethanol concentrations (10 min each in 50, 70, 80, 95% ethanol, followed by two 15 min dehydration in 100% ethanol) and two 10 min dehydrations in acetonitrile. The cell pellet was then infiltrated with 1:1 acetonitrile:araldite-epon overnight followed by 2 changes of aralide-epon and incubated overnight at room temperature. The blocks were polymerized for 2 d at 65° C. Thick sections were stained with toluidine blue, and thin sections were stained with 3% uranyl acetate in 30% ethanol for 15 min and by lead citrate for 2 min. The sections were examined in a JEOL 100CX or 100S electron microscope.

Epitope Chemical Analysis ELISAs. Several ELISA's were used to determine the nature of the epitopes recognized by the monoclonal antibodies. Whole cell M. tuberculosis was initially used which were treated with Sodium meta-periodate or proteinase K. The protocol for Sodium meta-periodate ELISA was adapted from the method of Udaykumar & Saxena (Udaykumar, and R. K. Saxena. Microbiol. Immunol. 76:7-12 (1991)). A 50 μl volume containing 1-2×10⁷ M. tuberculosis suspended in PBS was incubated in a microtiter polystyrene plate wells for 2 h at room temperature. After M. tuberculosis attached to the plate, the supernatant was removed and 50 μl of 0.1 M sodium meta-periodate (Sigma Chemical Co. St. Louis, Mo.) in 0.1 M acetate buffer (pH 4.5) was added to each well. Control wells had buffer only. The plates were incubated for 2 h at 4° C. in the dark, washed 5 times with 0.05% Tween 20 in PBS, and blocked with 200 μl 1% BSA in PBS. The plates were then used in ELISA to determine antibody binding to periodate treated M. tuberculosis. A similar ELISA procedure was done employing Proteinase K (Boehringer Manheim GmbH, Manheim, Germany) instead of sodium meta-periodate. Briefly, the plates were incubated with 100 μl of proteinase K at a concentration of 1 mg/ml in PBS or with PBS alone (as a control) at room temperature for 20 h and used as before.

ELISA was used to determine monoclonal antibody binding to mycobacterial fractions. Total lipid fraction (TLF) lipoarabinomannan (LAM), lipomannan (LM), mycolyl-arabinogalactan-peptidoglycan complex (mAGP—with protein contamination of 34 ng/mg) and phosphatidylinositol mannoside (PIM) from M. tuberculosis Erdman strain was kindly supplied by P. J. Brennan and J. T. Belisle (Department of Microbiology, Colorado State University, Fort Collins). The fractions were prepared from M. tuberculosis strain Erdman except for LM which was prepared from fast growing Mycobacterium sp..

The TLF ELISA used is a modification of protocols described previously (Cho, et al., J. Clin. Microbiol. 30:3065-3069 (1992)). TLF was suspended in 100% ethanol, added to polystyrene microtiter plates, serially diluted starting at a concentration of 1 mg/ml, and air dried overnight. The plates were then blocked with a solution of 1% BSA in PBS with 0.05% horse serum for 1.5 h at 37° C. and used to study monoclonal antibody binding to total lipid fraction by ELISA. Wells incubated with 100% ethanol without lipid antigen served as negative controls.

For the mycobacterial carbohydrate fraction ELISAs, a suspension of 100 μl mycobacterial antigens dissolved in carbonate buffer (pH 9.6) was placed in microtiter ELISA wells and incubated overnight at 4° C. (The concentrations of antigens LAM and mAGP were 10 μg/ml and 1 mg/ml respectively. LM and PIM were placed in serial dilutions starting at 50 μg/ml). Plates were then blocked with 3% BSA in PBS for 1.5 h at 37° C. After washing, 50 μl monoclonal antibodies solution (serial dilutions starting at 10 μg/ml for LAM and mAGP ELISAs and fixed concentration of 10 μg/ml for PIM and LM ELISAs) were added and ELISA procedure was followed as above. For comparative LAM-versus-LM ELISA 100 μl of antigen solution at 1 μg/ml were suspended in carbonate buffer (pH-9.6) and placed in microtiter ELISA plates. A 50 μl volume of relevant monoclonal antibody (5c11) was serially diluted across a microtiter plate starting at 10 μg/ml and the procedure was followed as above. Wells containing 50 μl carbonate buffer without antigen served as a control.

Western blot analysis. Whole cell M. tuberculosis Erdman strain were suspended in RIPA buffer (50 mM Tris Cl pH 7.5, 150 mM NaCl, 1% Nonidet P-40, 0.5% sodium deoxycholate, 0.1% SDS), frozen at −70° C., thawn, sonicated for 10 min and analyzed by sodium dodecyl sulfate polyacrylamide gel electrophoresis (SDS-PAGE) before and after reduction with β-mercaptoethanol in 12% gels. Gels were blotted onto nitrocellulose sheet and non specific binding sites were blocked with 3% gelatin in Tris buffered saline pH 7.5 (TBS) (BIO-RAD Laboratories, Hercules, Calif.). Blots were incubated overnight with either 10 μg/ml or 50 μg/ml monoclonal antibody diluted with 1% gelatin in 0.05% Tween in TBS (TTBS) at room temperature. After primary antibody incubation the blots were incubated with goat anti-mouse horseradish peroxidase-conjugated secondary antibody solution (BIO-RAD Laboratories, Hercules, Calif.) diluted 1:30 in 1% gelatin (BIO-RAD Laboratories, Hercules, Calif.). The blots were developed using color development reagents (BIO-RAD Laboratories, Hercules, Calif.) until the appearance of brown color. The positive control was an IgG1 monoclonal antibody to the 70 kD heat shock protein of M. tuberculosis.

Binding to other mycobacterial and bacterial strains. Comparative binding to other mycobacterial strains was done by whole cell ELISA and indirect immunofluorescence. Cells of mycobacterial and non-mycobacterial strains were suspended in PBS with 0.01 M sodium azide and washed twice. For whole cell ELISA mycobacterial and bacterial cells were resuspended to a turbidity value of 1 McFarland, placed in microtiter polystyrene plate and incubated overnight at 4° C. Plates were then blocked with 1% BSA in PBS with 0.05% horse serum 1.5 h at 37° C. After washing, 50 μl of monoclonal antibodies solution at 5 μg/ml was added, and the ELISA procedure was followed as described above.

For comparative indirect immunofluorescence, the cells of mycobacterial strains were suspended in PBS with 0.01 M sodium azide and washed twice with PBS. In addition to the standard strains, 3 clinical isolates of M. tuberculosis grown on LJ slants were tested. A 50 μl volume of a mycobacterial suspensions was placed on a poly-L-Lysine coated glass microscope slides (Poly-Prep slides, Sigma Diagnostics, St. Louis Mo.) and fixed by heating at 65° C. for 2 h. The immunofluorescence protocol was performed as described above using primary antibody at a concentration of 5 μg/ml and secondary FITC-labeled antibody at a concentration of 10 μg/ml. Negative controls consisted of incubating the various mycobacterial strains with FITC-labeled antibodies. The presence of mycobacteria on the slides was verified by acid-fast staining (performed on a separate slide).

Comparative binding of monoclonal antibody 9d8 to clinical strains of M. tuberculosis and M. avium-intracellulare complex.

Materials: 5 clinical isolates of M. tuberculosis grown on 7H10 solid media; 5 clinical isolates of MAC grown on 7H10 solid media; M. tuberculosis ERDMAN strain grown in PBT medium (control strain); glass slides; PBS with azide; monoclonal antibody 9d8; and FITC-labeled anti-mouse IgG.

Methods: Preparation of samples: Organisms were removed from medium with a sterile loop and suspended in 5 ml PBS with azide and washed. 3 drops (approximately 100 μl) were placed on a glass slide and fixed overnight at 70° C. Staining: Indirect immunofluorescence using 9d8. Staining was performed as described above, but not as a double stain. Positive controls: (1) Acid-fast stains of all strains performed in parallel to the experiment. (2) IF of M. tuberculosis ERDMAN strain grown in PBT medium (standard strain). Negative control: IF using FITC labeled IgG only.

Results: As shown in Table 1 below, 9d8 binds all clinical strains of M. tuberculosis; it does not bind 4 out of 5 MAC clinical strains. The IF of the 5th MAC was undetermined, and there was high background fluorescence not allowing a clear result. The presence of mycobacteria on the slide was determined by acid-fast stain performed in parallel.

TABLE 1

| Mycobacteria | Acid-Fast | IF |
|---|---|---|
| M. tuberculosis1 | + | + |
| M. tuberculosis-2 | + | + |
| M. tuberculosis-3 | + | + |
| M. tuberculosis-4 | + | + |
| M. tuberculosis-5 | + | + |
| M. tuberculosis-ERDMAN | | + |
| M. tuberculosis-FITC | | − |
| MAC-A | + | − |
| MAC-B | + | − |
| MAC-C | + | − |
| MAC-D | + | − |
| MAC-E | + | UD * |
| MAC-FITC | | − |

* UD - undetermined

Testing the protective potential of monoclonal antibody 9d8 in a mouse model.

Experiment 1: Mice: C57BL/6 mice were divided into experimental and control groups. Monoclonal antibody administration: The experimental group received ascites containing monoclonal antibody 9d8 at a concentration of 91 μg/ml. The control group received NSO ascites. One ml of ascites was injected intraperitoneally 3 times at one week intervals. Infection: Both groups were infected intravenously with 1×10⁴ M. tuberculosis Erdman strain. M. tuberculosis was injected 4 hours after the first ascites administration. Mice from each group were sacrificed after 3 weeks. Lung, spleen and liver were harvested for CFUs.

Experiment 2: Mice: C57BL/6 mice were divided into 3 groups. Infection: M. tuberculosis Erdman strain was administered intratracheally. An estimated number of 5000 organisms were used in each intratracheal injection. Monoclonal antibody administration: Monoclonal antibody 9d8 at a concentration of 91 μg/ml was administered in one of two ways: Mice group 1: 1 cc of monoclonal antibody in the form of ascites injected intraperitoneally every 24 hours for 3 days. 3^{rd} injection was administered 4 hours prior to M. tuberculosis inoculation. A 4^{th} dose of monoclonal antibody was administered the day after M. tuberculosis inoculation. Mice group 2: A mixture of monoclonal antibody 9d8 (in the form of ascites) and M. tuberculosis (incubated at room temperature for 2 hr and washed to remove unbound material) administered intratracheally. Mice group 3 (control): NSO ascites (1 ml) was administered intraperitoneally at the same schedule as monoclonal antibody 9d8 that was administered to group 1. Mice were sacrificed at the following schedule: 24 hours and 3 weeks. A subset of mice were observed for monoclonal antibody effects on survival. The original intratracheal inocula were plated for accurate colony count.

B. Results and Discussion

Isolation of hybridomas producing anti-M. tuberculosis antibodies. A single fusion was performed using the spleen from the mouse that raised the highest antibody titer against whole cell M. tuberculosis (titer was 2187 fold over background, prior to last boosting). A total of 1152 wells were seeded with fused NSO-myeloma-splenocytes and their supernatants were screened by whole cell M. tuberculosis ELISA 8 d after fusion. A total of 25 wells had optical density of >0.4 at 405 nm. After 2 cloning procedures in soft agar 3 stable clones (5c11, 9d8 and 4f11) were obtained. Isotype determination by ELISA with goat anti-mouse isotype specific reagents revealed that one clone (9d8) secreted IgG3 and two clones (5c11 and 4f11) secreted IgM. All 3 monoclonal antibodies had kappa isotype light chain.

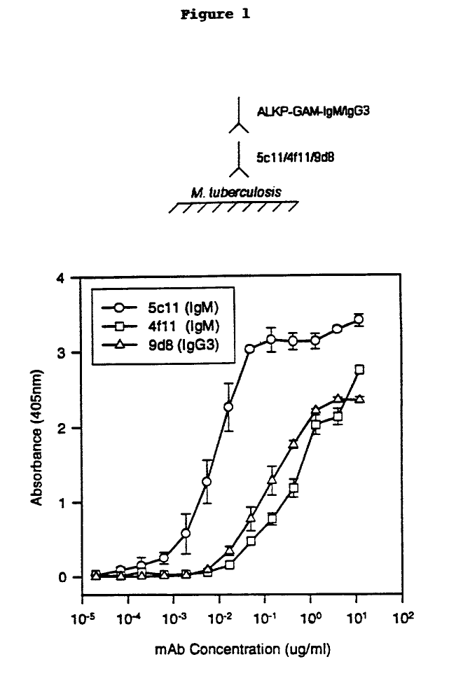

M. tuberculosis whole cell ELISA. All 3 monoclonal antibodies bound to plates coated with whole M. tuberculosis by ELISA. Comparative binding of the 3 monoclonal antibodies was performed by serially diluting the monoclonal antibodies. The binding curves show that monoclonal antibody 5c11 (IgM) required 10 to 15 times lower concentration than monoclonal antibodies 4f11 (IgM) or 9d8 (IgG3) to achieve the same optical density signal (FIG. 1). This difference was maintained even at very low optical density signals. This suggests either a higher binding affinity for 5c11 or a higher prevalence of 5c11 epitopes on the surface of M. tuberculosis.

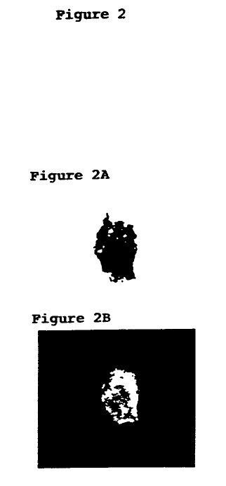

Indirect Immunofluorescence. All 3 monoclonal antibodies showed strong indirect immunofluorescence after incubation with whole cell M. tuberculosis. The fluorescence intensity was strongest at monoclonal antibody concentrations of 1-10 μg/ml and faded at monoclonal antibody concentration between 0.1 and 0.01 μg/ml (see Table 2). An acid-fast staining prior to the addition of monoclonal antibodies and FITC conjugated antibodies, had little or no effect on the fluorescence intensity (FIG. 2).

TABLE 2
Immunofluorescence endpoints demonstrating signal
intensity at various monoclonal antibody concentrations

| | 10 | 1 | 0.1 | 0.01 | 0.001 |
|---|---|---|---|---|---|
| | μg/ml | μg/ml | μg/ml | μg/ml | μg/ml |
| 5c11 | +++ | ++ | +_{W} | − | − |
| (IgM) | | | | | |
| 4f11 | ++ | ++ | +_{W} | − | − |
| (IgM) | | | | | |
| 9d8 | +++ | ++ | + | − | − |
| (IgG3) | | | | | |

W - weak

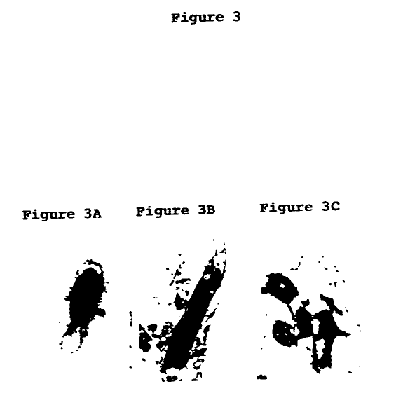

Immunoelectronmicroscopy. The binding of each monoclonal antibody to M. tuberculosis was studied by immunoelectronmicroscopy. Mycobacterial cell wall architecture was preserved but cytoplasmic mycobacterial structures could not be clearly identified due to the prolonged heat killing. Gold particles appeared to concentrate on the surface of the organism at or outside the level of the outer layer for each of the 3 monoclonal antibodies specimens (FIG. 3). Localization of gold particles to cell wall structures is consistent with the results of whole cell ELISA and immunofluorescence.

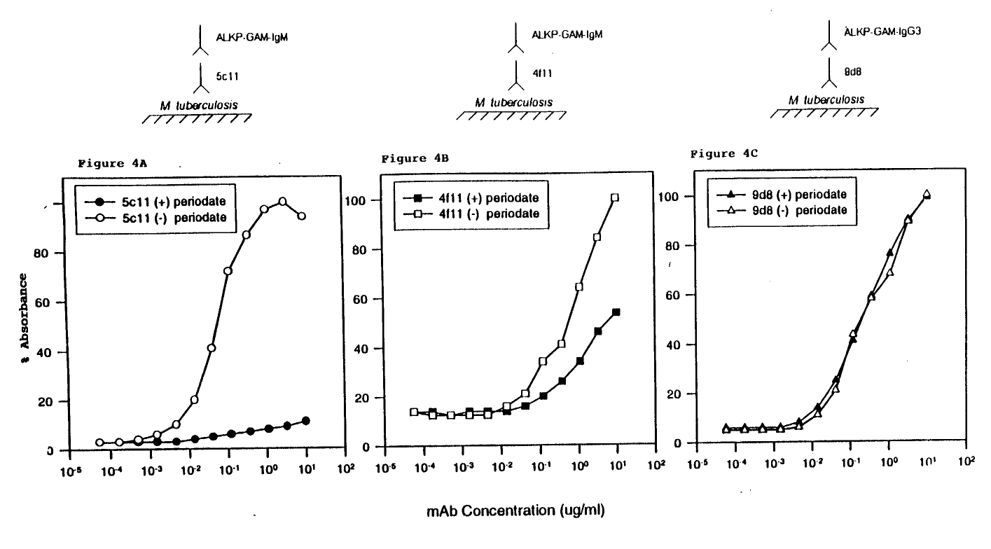

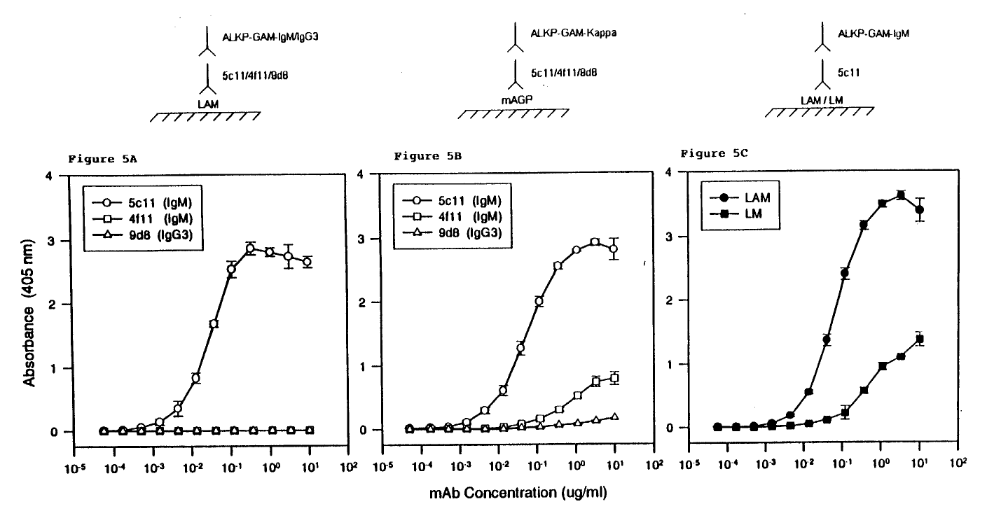

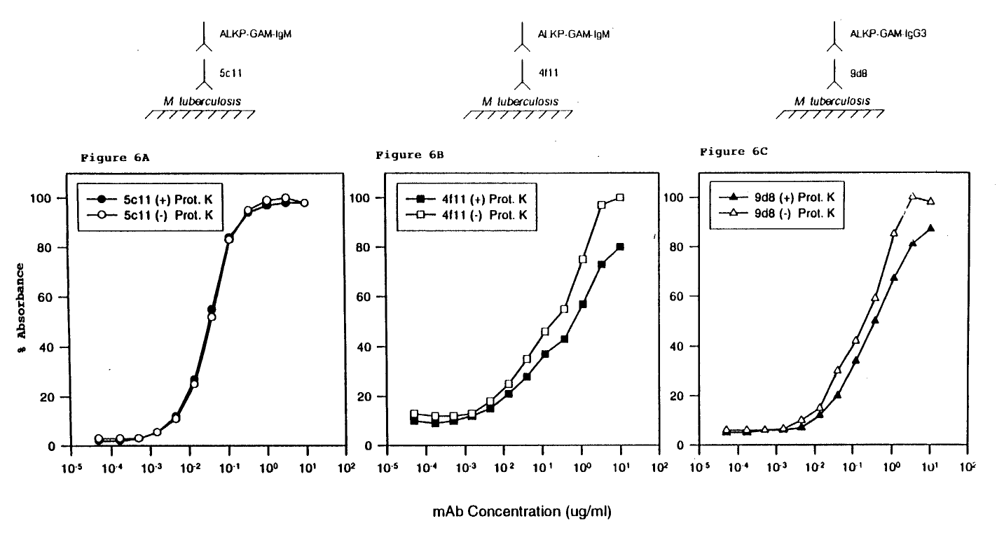

Epitope Chemical Analysis ELISAs. Sodium meta-periodate at acid pH causes mild oxidation of carbohydrate hydroxyl groups and opens sugar rings (Watt, et al., J Infect Dis. 158:681-686 (1988)). Treatment of whole cell M. tuberculosis with sodium meta-periodate resulted in reduced binding of monoclonal antibodies 5c11 and 4f11 to whole cell M. tuberculosis (FIG. 4) consistent with the presence of carbohydrates in the monoclonal antibodies epitopes. ELISAs performed with specific cell wall carbohydrates revealed that monoclonal antibodies 5c11 and 4f11 bound to mAGP (FIG. 5B) while only monoclonal antibody 5c11 bound LAM (FIG. 5A). Monoclonal antibody 5c11 bound significantly stronger to LAM than to LM at a monoclonal antibody concentration of 1 μg/ml (FIG. 5C). Proteinase K treatment of whole cell M. tuberculosis reduced the binding of monoclonal antibodies 9d8 and 4f11 but did not affect the binding of monoclonal antibody 5c11 (FIG. 6). None of the monoclonal antibodies bound PIM or TLF by ELISA.

Western blot analysis. None of the monoclonal antibodies reacted with mycobacterial antigens by Western Blot analysis while the control monoclonal antibody to M. tuberculosis 70 kD heat shock protein showed a clear band.

Binding to other mycobacterial strains. Two methods were used for comparing monoclonal antibody binding to other mycobacterial strains: whole cell ELISA and indirect immunofluorescence. By whole cell ELISA both IgM monoclonal antibodies (5c11 and 4f11) bound to multiple mycobacterial strains. IgG3 monoclonal antibody 9d8 was more selective than the other monoclonal antibodies. In addition to binding the surface of M. tuberculosis, monoclonal antibody 9d8 also bound to M. gordonae, M. gastri, and M. kansasii. Indirect immunofluorescence demonstrated a similar trend. (Table 3).

TABLE 3
Binding of monoclonal antibodies to various
mycobacterial and non-mycobacterial strains

| | 5c11 (IgM) | | 9d8 (IgG3) | | 4f11 (IgM) | |
|---|---|---|---|---|---|---|
| | ELISA* | IF | ELISA* | IF | ELISA* | IF |
| M. tuberculosis #1 | 1.000 | +++ | 1.000 | ++ | 1.000 | +++ |
| M. tuberculosis #2 | 0.490 | ++ | 0.237 | ++ | 0.393 | ++ |
| M. bovis-BCG | 0.614 | +++ | 0.032 | − | 0.267 | +++ |
| M. microti | 0.493 | ++ | 0.160 | − | 0.166 | ++ |
| M. avium | 0.175 | ++ | 0.013 | − | 0.070 | ++ |
| M. smegmatis | 0.491 | +_{W} | 0.036 | − | 0.414 | I |
| M. xenopi | 0.603 | ++ | 0.035 | − | 1.397 | ++ |
| M. chitae | 0.243 | ++ | 0.039 | − | 0.125 | + |
| M. marinum | 0.207 | + | 0.055 | − | 0.091 | − |
| M. chelonae | 0.377 | ++ | 0.050 | − | 0.199 | +++ |
| M. gastri | 0.720 | ++ | 0.710 | ++ | 0.975 | ++ |
| M. kansasii | 0.535 | ++ | 0.485 | + | 0.606 | + |
| M. vaccae | 0.368 | ++ | 0.017 | − | 0.044 | + |
| M. phlei | 0.592 | ++ | 0.027 | − | 0.096 | ++ |
| M. fortuitum | 1.054 | + | 0.096 | − | 1.237 | + |
| M. terrae | 0.092 | + | 0.105 | − | 0.318 | + |
| M. szulgai | 0.836 | ++ | 0.163 | − | 2.090 | ++ |
| M. gordonae | 0.879 | ++ | 0.955 | + | 1.871 | ++ |
| Strep. pneumo. | 0.000 | ND | 0.000 | ND | 0.002 | ND |
| E. coli | 0.000 | ND | 0.001 | ND | 0.017 | ND |
| Coryneba. pseud. | 0.002 | ND | 0.009 | ND | 0.009 | ND |
| N. asteroides | 0.049 | ND | 0.077 | ND | 0.109 | ND |
| P. aeruginosa | 0.000 | ND | 0.000 | ND | 0.000 | ND |
| H. influenzae | 0.001 | ND | 0.017 | ND | 0.031 | ND |

*ELISA comparison was done with optic density ratio using M. tuberculosis Erdman strain grown in PBT medium as reference.
IF - Indirect immunofluorescence.
1 - M. tuberculosis Erdman strain grown in PBT medium.
2 - M. tuberculosis Erdman strain grown in LJ medium. IF using 3 clinical strains of M. tuberculosis grown in LJ medium gave similar results.
I - Indeterminate.
ND - Not done.
W - Weak.

Experiment 1: Table 4 demonstrates that mice treated with monoclonal antibody 9d8 intraperitoneally had lower CFUs than mice treated with NSO ascites. This effect was statistically significant in the liver as compared to mice receiving NSO ascites (p-value 0.0420).

TABLE 4
CFU counts in mice sacrificed 3 weeks after M. tuberculosis challenge

| | Spleen (5 ml) | Liver (10 ml) | Lungs (5 ml) |
|---|---|---|---|
| 9d8 (IgG3) | 9999^{b} | 120000 | 2660000 |
| 9d8 (IgG3) | 9999^{b} | 19999^{b} | 9999^{b} |
| 9d8 (IgG3) | 9999^{b} | 20000 | 75000 |
| 9d8 (IgG3) | 780000 | 100000 | 9999^{b} |
| 9d8 (IgG3) | 846666 | 19999^{b} | 9999^{b} |
| 9d8 (IgG3) | 140000 | 40000 | 9999^{b} |
| 9d8 (IgG3) | 30000 | 40000 | 9999^{b} |
| Mean | 260951 | 51428 | 397856 |
| NSO ascites | 245000 | 80000 | 450000 |
| NSO ascites | 280000 | 866666 | 96433333 |
| NSO ascites | 385000 | 150000 | 200000 |
| NSO ascites | 905000 | 160000 | 9999^{b} |
| NSO ascites | 9999_{b} | 300000 | 9999^{b} |
| NSO ascites | 1006666 | 19999^{b} | 140000 |
| Mean | 471944 | 262777 | 16207221 |
| p-value (2 | 0.1984 | 0.0420 | 0.2158 |
| sample T-test) | | | |

^{b}No colonies were counted on the plate, but due to a dilution factor, there is a theoretical possibility of having up to 9999 or 19999 colonies (depending on the dilution).

Experiment 2: CFU'S: There was no statistical significance in the CFU counts in these experiments: (6 mice from each group were sacrificed at 3 weeks, 3 mice from each monoclonal antibody group and 1 mouse from the control group were sacrificed at 24 hours).

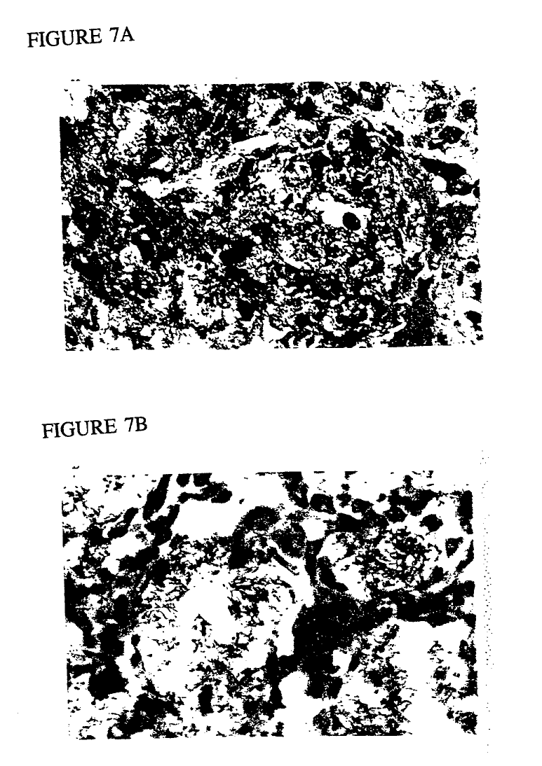

Pathology: A significant difference was demonstrated in lung pathology (FIGS. 7A and 7B). In the lungs of mice treated with a mixture of M. tuberculosis and monoclonal antibody 9d8, (group 2) infection appeared more controlled and organized: there were more granulomas, acid-fast bacilli were limited to granulomatous areas and were circumscribed by them. In the lungs of mice treated with NSO ascites (group 3), acid-fast bacilli appeared more dispersed throughout the tissue, with less granuloma formation. Even when granulomas were present, they appeared less organized (fewer layers of cells, less defined).

FIGS. 7A and 7B represent the differences: FIG. 7A demonstrates lung tissue from a mouse that received NSO ascites (group 3). Acid-fast bacilli are dispersed throughout the tissue, cells are not well organized in granulomas. FIG. 7B shows lung tissue from a mouse that received a mixture of M. tuberculosis and monoclonal antibody 9d8 (group 2). Acid fast bacilli are contained in well organized granulomas. The best organization is represented by few layers of cells circumscribing acid-fast bacilli. A similar picture to that of the control lungs (FIG. 7A) was seen in lungs of mice treated with monoclonal antibody 9d8 intraperitoneally (group 1). (Exact quantitation of the phenomenon is difficult because there is a spectrum of manifestations. In addition, unless the same exact lung area is compared in each mouse, exact numbers are meaningless).

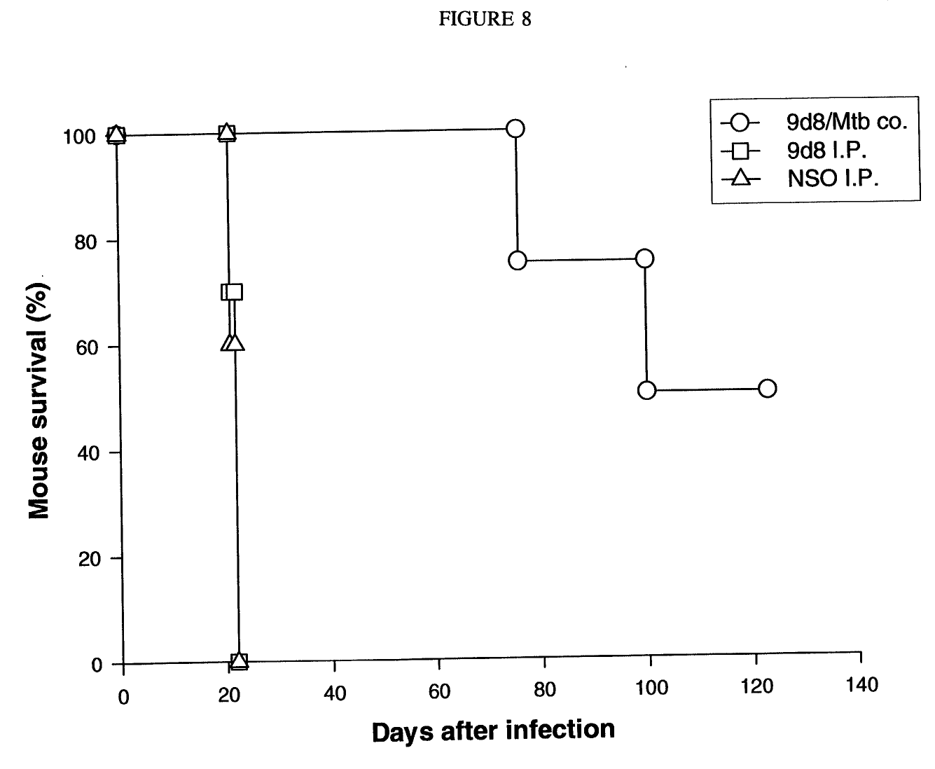

Survival: Significant differences were seen in survival of mice (FIG. 8). Mice treated with a mixture of M. tuberculosis/monoclonal antibody 9d8 (round symbols) survived longer than mice from the other 2 groups: 1 mouse died on day 76, the second mouse dies at 100 days, and 2 mice were sacrificed on day 123. Mice treated with NSO ascites and 9d8 ascites intraperitoneally, died by day 22 (red and green lines). The lungs of the 2 mice that were sacrificed on day 123 showed signs of healing.

Inocula CFU's: Mixture of monoclonal antibody 9d8 and M. tuberculosis: 2900; M. tuberculosis alone: 4450; which is comparable.

Discussion

All 3 monoclonal antibodies generated in this study bound to the surface of M. tuberculosis as demonstrated by whole cell ELISA, indirect immunofluorescence and immunoelectronmicroscopy. The results of binding studies with defined mycobacterial fractions, suggest that monoclonal antibodies 5c11 and 4f11 bind epitopes containing carbohydrates. Monoclonal antibody 5c11 binds both LAM and LM, but the stronger affinity for LAM relative to LM by ELISA suggests that the arabinose moiety is an important part of the epitope recognized. Both 5c11 and 4f11 bind to mAGP—which is a fraction of the mycobacterial cell wall left after removing all soluble carbohydrates, proteins, and lipids (Wu, et al., Chin. J. Microbiol. Immunol. 22:173-180 (1989)). The strong binding of 5c11 to this complex is consistent with either the presence of LAM in the preparation or binding to arabinose which is found also in the mAGP complex. mAGP is known to be associated with protein in the mycobacterial cell wall skeleton in a complex called mycolyl-arabinogalactan-peptidoglycan-protein (mAGPP) (Wu, et al., Chin. J. Microbiol. Immunol. 22:173-180 (1989)). The reduction in binding of monoclonal antibody 4f11 to proteinase K-treated M. tuberculosis suggests that proteinase K digestion removed or destroyed part of the epitope recognized by this monoclonal antibody. For monoclonal antibody 9d8 no direct evidence was found for binding to protein, carbohydrate or lipid antigen. However, treatment of mycobacteria with proteinase K also reduced monoclonal antibody 9d8 binding, suggesting that the 9d8 epitope either contains or is attached to a protein moiety. No evidence for monoclonal antibody binding to protein was obtained by Western blot analysis for any of the 3 monoclonal antibodies. Hence, it may be concluded that monoclonal antibody 5c11 binds LAM, 4f11 binds a cell wall carbohydrate that belongs to the mAGP complex, and 9d8 binds a cell wall epitope of an uncertain composition which contains protein or is associated with protein. The results suggest however that protein is not a major component of the epitope recognized by monoclonal antibody 9d8.

The reactivity of the 3 monoclonal antibodies with 17 mycobacterial and 6 non-mycobacterial species was investigated. Monoclonal antibodies 9d8 and 5c11 were the most and least selective respectively, in their reactivity with different mycobacterial species. The low selectivity of monoclonal antibody 5c11 can be explained by the fact that most, if not all, mycobacterial strains contain LAM. None of the monoclonal antibodies bound to non-mycobacterial bacterial species. When interpreting the data in Table 3 it is important to consider that inter-species comparisons are difficult because there are differences in the adherence of mycobacterial species to polystyrene. This is not a problem for intra-species comparisons of 5c11, 9d8 and 4f11 binding. The ELISA and immunofluorescence binding results parallel each other for the majority of mycobacterial species. For some strains such as M. avium, immunofluorescence and ELISA reactivity are significantly different. This problem is not understood but may reflect differences in epitope availability for mycobacteria attached to polystyrene or glass. The differences in monoclonal antibodies 5c11, 9d8 and 4f11 with individual strains are consistent with recognition of different epitopes by each monoclonal antibody.

Further, the experiments suggest that monoclonal antibody 9d8 (IgG3) has a role in reducing CFU's in organs of C57B1/6 mice that received an intravenous infection (as demonstrated by Experiment No. 1). Monoclonal antibody 9d8 also affects survival of mice and lung pathology when administered intratracheally (as demonstrated in Experiment No. 2). The implication of this experiment is that antibodies may indeed have a role in protection against tuberculosis. The effect does not appear to be due to neutralization effect because inocula CFU's were comparable. Monoclonal antibody 9d8 defines an epitope that could elicit protective antibodies. Hence this monoclonal antibody may be used to isolate the epitope and generate a novel type of vaccine.

All publications mentioned hereinabove are hereby incorporated by reference in their entirety.

While the foregoing invention has been described in detail for purpose of clarity and understanding, it will be appreciated by one skilled in the art from a reading of the disclosure that various changes in form and detail can be made without departing from the true scope of the invention in the appended claims.

What is claimed is:

1. A method for treating M. tuberculosis infection in a subject comprising administering an amount of a monoclonal antibody effective to treat infection of M. tuberculosis in a subject wherein the antibody binds to the cell surface of M. tuberculosis and is not cross-reactive with M. avium-intracellulare and M. bovis-BCG.

2. The method of claim 1 wherein the monoclonal antibody recognizes a non-protein epitope on the cell surface of M. tuberculosis.

3. The method of claim 1 wherein the monoclonal antibody binds to the same antigen as the monoclonal antibody produced by hybridoma cell line H-9d8 having ATCC Accession No. ______.

4. A purified determinant that elicits antibodies that bind to the same cell surface epitope of M. tuberculosis to which monoclonal antibody 9d8 binds.

5. A method for protecting a subject against tuberculosis comprising administering an amount of the antigenic determinant of claim 4 effective to protect a subject against tuberculosis.
